(19) United States
(12) Patent Application Publication
(10) Pub. No.: US 2003/0211105 A1
(43) Pub. Date: Nov. 13, 2003

(54) Methods for reducing tumor growth and metastasis by inhibiting mcp-1 activity

(76) Inventors: William J Murphy, Reno, NV (US); Joost J Oppenheim, Bethesda, MD (US); Rosalba Salcedo, Frederick, MD (US)

Correspondence Address: NEEDLE & ROSENBERG, P.C., SUITE 1000, 999 PEACHTREE STREET, ATLANTA, GA, 30309-3915, US

(21) Appl. No.: 10/276,644

(22) PCT Filed: May 18, 2001
(86) PCT No.: PCT/US01/16058

(51) Int. Cl. A61K 31/00; C12Q 1/68; G01N 33/574; A61K 39/395

(52) U.S. Cl. 424/146.1; 514/1; 435/6; 435/7.23

(57) ABSTRACT

The present invention features methods of inhibiting tumor growth and/or tumor metastasis in a subject, and methods of treating cancer and/or increasing survival of a subject with a tumor, by inhibiting MCP-1 activity in the subject. The present invention also features methods of identifying a compound that inhibits tumor growth or metastasis, by exposing a sample including an MCP-1 polypeptide, an MCP-1 nucleic acid, an MCP-1 receptor polypeptide, or an MCP-1 receptor nucleic acid to a test compound, and detecting a decrease in MCP-1 activity in the sample, wherein a decrease in MCP-1 activity in the sample identifies a compound that inhibits tumor growth or tumor metastasis.

### CROSS REFERENCE TO RELATED APPLICATION

This application claims priority to U.S. Ser. No. 60/205,757, filed May 19, 2000, herein incorporated by reference.

### ACKNOWLEDGEMENTS

This invention was made with intramural support from the National Institutes of Health. The government has certain rights to the invention.

### FIELD OF THE INVENTION

This invention relates generally to methods for inhibiting tumor growth and metastasis, specifically, by inhibiting Monocyte Chemotactic Protein 1 (MCP-1) activity.

### BACKGROUND OF THE INVENTION

Monocyte Chemotactic Protein 1 (MCP-1) is a chemokine that is abundantly produced in a variety of inflammatory diseases (Cushing et al., Proc. Natl. Acad. Sci. USA 87:5134-5138, 1990; Koch et al., J. Clin. Invest. 90:772-779, 1992). Consistent with its role in inflammation, MCP-1 is known to be chemotactic for monocytes, T lymphocytes, basophils, and NK cells.

Based on its chemotactic effect on monocytes, MCP-1 has been observed to have an anti-tumor effect in certain mouse/tumor experimental systems (Zhang et al., J. Immunol. 158:4855-4861 (1997); Zhang et al., Lab. Invest. 76:579-590 (1997); Hoshino et al., Exp. Hematol. 23:1035-1039 (1995)). In those mouse systems, MCP-1 production by tumor cells was positively correlated with the number of intratumoral macrophages and inversely correlated with tumor growth. These studies have led to the hypothesis that MCP-1 possesses anti-tumorigenic activity.

The present invention provides two surprising discoveries: first, that MCP-1 is directly chemotactic for vascular endothelial cells, and, thus, is a direct stimulator of angiogenesis; second, and contrary to what would be expected, that inhibition of MCP-1 activity inhibits tumor metastasis and prolongs survival of immunodeficient mice carrying human tumors. As most human cancers are not subject to growth inhibition by the human immune system under normal conditions, the observations described herein using the present mouse/tumor system more closely reflect the physiological situation in most human cancers. Because tumor growth and metastasis in humans is stimulated by tumor-associated angiogenesis, the surprising discovery that inhibition of MCP-1 decreases tumor metastasis and increases survival provides much-needed new approaches for cancer treatment.

### SUMMARY OF THE INVENTION

The present invention is based on the surprising discovery, described herein, that inhibition of MCP-1 activity inhibits tumor metastasis and prolongs survival.

In a first aspect, the invention features a method of inhibiting metastasis of a tumor in a subject. The method includes inhibiting MCP-1 activity in the subject, thereby inhibiting metastasis of the tumor in the subject.

In a second aspect, the invention features a method of inhibiting growth of a tumor in a subject. The method includes inhibiting MCP-1 activity in the subject, thereby inhibiting growth of the tumor in the subject.

In a third aspect, the invention features a method of increasing the survival of a subject with a tumor. The method includes inhibiting MCP-1 activity in said subject, thereby increasing survival of the subject.

In one embodiment of the first three aspects of the invention, the tumor produces MCP-1. In another embodiment of the first three aspects of the invention, the tumor is a solid tumor, for example, and not limited to, a breast tumor (e.g., a breast carcinoma); a melanoma; a colon carcinoma; a lung tumor (e.g., a lung carcinoma); a kidney carcinoma; a liver carcinoma; a uterine tumor (e.g., an endometrial or cervical carcinoma or a leiomyoma); an ovarian carcinoma; an osteosarcoma, or a tumor of the nervous system, such as a glioblastoma or a neuroblastoma. In still another embodiment of the first three aspects of the invention, the tumor metastasizes in the subject, for example, to a lung in the subject.

In a fourth aspect, the invention features a method of treating cancer in a subject. The method includes inhibiting MCP-1 activity in the subject, wherein inhibiting MCP-1 activity in the subject inhibits tumor growth or metastasis in the subject, thereby treating cancer in the subject.

In a preferred embodiment of the fourth aspect of the invention, the cancer may be a leukemia or a lymphoma, or the cancer may be a solid tumor, for example one of the solid tumors listed above. In addition, the method of the fourth aspect of the invention is useful for treating any cancer that produces MCP-1.

In a preferred embodiment of the first four aspects of the invention, the subject is a mammal, for example, a human. The mammal may also be a laboratory animal, such as a mouse (e.g., a SCID mouse) or other rodent (e.g., a rat or a guinea pig), or any other mammal used for biomedical experimentation, e.g., a monkey or an ape, a dog, a pig, a rabbit, or a goat. The mammal may also be a domestic or commercially valuable animal, such as a monkey or an ape, dog, a cat, a horse, a goat, a sheep, or a cow, or any genetically modified mammal.

In another preferred embodiment of the first four aspects of the invention, the tumor includes human tumor cells.

In other preferred embodiments of the first four aspects of the invention, the MCP-1 activity that is inhibited is the stimulatory activity of MCP-1 on: angiogenesis, cell proliferation, tumor growth, and/or tumor metastasis. A cell that is stimulated to proliferate by MCP-1 may be a vascular endothelial cell or a tumor cell (e.g., a breast tumor cell).

In another preferred embodiment of the first four aspects of the invention, MCP-1 activity is inhibited in the subject by inhibiting the interaction between MCP-1 and an MCP-1 receptor in the subject. For example, the MCP-1 receptor may be contacted with an antibody that specifically binds to the MCP-1 receptor, thereby inhibiting MCP-1 activity in the subject. The receptor may be present, for example, on the surface of a vascular endothelial cell or on the surface of a tumor cell. The antibody may be a polyclonal antibody or a monoclonal antibody.

In yet another preferred embodiment of the first four aspects of the invention, the MCP-1 activity may be inhibited by administering to the subject an antibody that specifically interacts with MCP-1; such an antibody may be a polyclonal antibody or a monoclonal antibody.

In other preferred embodiments of the first four aspects of the invention, the MCP-1 activity may be inhibited by contacting an MCP-1 polypeptide molecule or an MCP-1 receptor polypeptide molecule with an MCP-1 inhibitory molecule isolated by combinatorial chemistry or in vitro selection, or the MCP-1 activity may be inhibited by contacting an MCP-1 nucleic acid molecule or an MCP-1 receptor nucleic acid molecule with an MCP-1 inhibitory molecule isolated by combinatorial chemistry or in vitro selection.

In yet other preferred embodiments of the first four aspects of the invention, the MCP-1 activity may be inhibited by delivering a functional nucleic acid to an MCP-1 producing cell within the subject. The functional nucleic acid may specifically interact with an MCP-1 polypeptide molecule or with an MCP-1 nucleic acid molecule, and the functional nucleic acid may be selected from the group consisting of an antisense nucleic acid, a catalytic nucleic acid, an aptamer, and an external guide sequence.

In a fifth aspect, the invention features a method of identifying a compound that inhibits tumor growth or tumor metastasis. The method includes the steps of: a) exposing a sample including an MCP-1 polypeptide or an MCP-1 nucleic acid to a test compound; and b) detecting a decrease in MCP-1 activity in the sample, wherein a decrease in MCP-1 activity in the sample identifies a compound that inhibits tumor growth or tumor metastasis.

In a sixth aspect, the invention features a method of identifying a compound that inhibits tumor growth or tumor metastasis. The method includes the steps of: a) exposing a sample including an MCP-1 receptor polypeptide or an MCP-1 receptor nucleic acid to a test compound; and b) detecting a decrease in MCP-1 activity in the sample, wherein a decrease in MCP-1 activity in the sample identifies a compound that inhibits tumor growth or tumor metastasis.

In a seventh aspect, the invention features a method of identifying a compound that inhibits tumor growth or tumor metastasis. The method includes the steps of: a) exposing a sample including an MCP-1 molecule and an MCP-1 receptor molecule to a test compound; and b) detecting specific binding of the MCP-1 molecule to the MCP-1 receptor molecule in the sample, wherein inhibition of specific binding of the MCP-1 molecule to the MCP-1 receptor molecule identifies a compound that inhibits tumor growth or tumor metastasis.

In an eighth aspect, the invention features a method of identifying a compound that increases survival of a subject with a tumor. The method includes the steps of: a) exposing a sample including an MCP-1 polypeptide or an MCP-1 nucleic acid to a test compound; and b) detecting a decrease in MCP-1 activity in the sample, wherein a decrease in MCP-1 activity in the sample identifies a compound that increases survival of a subject with a tumor.

In a ninth aspect, the invention features a method of identifying a compound that increases survival of a subject with a tumor. The method includes the steps of: a) exposing a sample including an MCP-1 receptor polypeptide or an MCP-1 receptor nucleic acid to a test compound; and b) detecting a decrease in MCP-1 activity in the sample, wherein a decrease in MCP-1 activity in the sample identifies a compound that increases survival of a subject with a tumor.

Additional advantages of the invention will be set forth in part in the description which follows, and in part will be apparent from the description, or may be learned by practice of the invention. It is to be understood that both the foregoing general description and the following detailed description are exemplary and explanatory only and are not restrictive of the invention as claimed.

### DETAILED DESCRIPTION OF THE INVENTION

MCP-1, a member of the CC chemokine family, is known to be directly chemotactic for cells involved in inflammatory processes, i.e., monocytes, T lymphocytes, basophils, and NK cells. The present invention provides two surprising findings: first, that MCP-1 is directly chemotactic for endothelial cells, and thus, is a direct mediator of angiogenesis; second, that inhibition of MCP-1 activity unexpectedly inhibits tumor growth and metastasis and prolongs survival in immunodeficient mice carrying human tumors.

As described herein, MCP-1 induced endothelial cell migration in a dose-responsive manner in endothelial cells of two different origins (human umbilical cord vein endothelial cells (HUVECs) from neonatal umbilical cords and human dermal microvascular endothelial cells (HMECs)). This chemotactic response was inhibited by a neutralizing antibody to MCP-1. The ability of HUVECs and HMECs to respond to MCP-1 was consistent with the presence of CCR2 (the receptor for MCP-1) on the endothelial cell surface. The angiogenic effect of MCP-1 was clearly evident in both in vivo Matrigel™ plug assays and chorioallantoic membrane (CAM) assays, and was appropriately inhibited by a neutralizing antibody against MCP-1. This effect, therefore, was consistent with the expression of CCR2 by endothelial cells and their responsiveness towards MCP-1.

Because inflammation can induce angiogenesis, rat aortic ring assays were used to evaluate angiogenic effects in the absence of an inflammatory response; these assays showed that MCP-1 induced rat aortic endothelial cell sprouting in a dose-responsive manner. Thus, MCP-1 can act as a direct mediator of angiogenesis.

Tumor-associated angiogenesis stimulates tumor growth and metastasis, and leads to a poor prognosis for patients in which such tumor-associated angiogenesis occurs. To test whether MCP-1 is involved in stimulation of tumor growth and metastasis, CB-17 severe combined immune deficient (SCID) mice treated with an anti-ASGM1 antibody (and, therefore, lacking T cells, B cells, and NK cells, thereby eliminating confounding effects of the immune system), were used in the studies of tumor growth and metastasis described herein.

As described herein, treatment of SCID mice bearing human breast carcinoma cells with a neutralizing antibody against MCP-1 significantly enhanced survival of the mice. Moreover, growth of tumor metastases in the lungs of anti-MCP-1 antibody-treated mice was inhibited by about 2.5-fold, compared with control antibody-treated mice. Thus, inhibition of MCP-1 activity provides a promising new approach for the treatment and prevention of metastastic cancer.

Definitions

In this specification and in the claims that follow, reference is made to a number of terms that shall be defined to have the following meanings. It is to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a pharmaceutical carrier” can mean a single pharmaceutical carrier or mixtures of two or more such carriers.

By “about” is meant ±10% of a recited value. Ranges may be expressed herein as from “about” one particular value, and/or to “about” another particular value. Similarly, when values are expressed as approximations, by use of the antecedent “about,” it will be understood that the particular value constitutes another embodiment. It will be further understood that the endpoints of each of the ranges are significant both in relation to the other endpoint, and independently of the other endpoint.

By “MCP-1 polypeptide” is meant a polypeptide that binds to an MCP-1 receptor (e.g., a CCR2 receptor) on vascular endothelial cells and induces vascular endothelial cell chemotaxis, as described herein. An MCP-1 polypeptide contains an amino acid sequence that bears at least 80% sequence identity, preferably at least 85% sequence identity, more preferably at least 90% sequence identity, still more preferably at least 95%, 96%, 97%, 98%, or 99% sequence identity, and most preferably 100% sequence identity with an MCP-1 amino acid sequence known in the art. MCP-1 is encoded by a single gene which is well conserved in several species, including human, mouse, and rat (Yoshimura et al., FEBS Lett. 244:487-493, 1989 and Timmers et al., Nucleic Acids Res. 18:23-34, 1990). Examples of MCP-1 polypeptides include, but are not limited to, the human, mouse, rat, porcine, and bovine MCP-1 polypeptides set forth in Yoshimura et al, supra; Timmers et al, supra; Hosang et al., Biochem. Biophys. Res. Commun. 199:962-968, 1994; and Wempe et al., DNA Cell Biol. 10:671-679, 1991. MCP-1 is also known in the art as “MCAF” (monocyte chemotactic and activating factor) and “JE”.

By “MCP-1 nucleic acid” is meant a nucleic acid that encodes an MCP-1 polypeptide, as defined above.

By “MCP-1 receptor polypeptide” is meant a receptor present on the surface of a cell (e.g., a vascular endothelial cell) that binds MCP-1 and mediates MCP-1 activity (e.g., MCP-1-specific endothelial cell chemotaxis). One example of an MCP-1 receptor is the human MCP-1 receptor known as “CCR2,” which is described in Charo et al., Proc. Natl. Acad. Sci. U.S.A. 91:2752-2756, 1994. Another example of an MCP-1 receptor is the murine MCP-1 receptor (also known as “mCCR2”) described in Kurihara and Bravo, J. Biol. Chem. 271:11603-11607, 1996.

By “MCP-1 receptor nucleic acid” or “CCR2 nucleic acid” is meant a nucleic acid that encodes an MCP-1 receptor polypeptide, as defined above.

By “MCP-1 activity” is meant any measurable physiological function attributable to an MCP-1 polypeptide, including (but not limited to) any one or more of: binding of MCP-1 to its cognate receptor (e.g., the CCR2 receptor); MCP-1-dependent stimulation of MCP-1 receptor dimerization; MCP-1-dependent stimulation of chemotaxis by monocytes, T lymphocytes, basophils, NK cells, endothelial cells, and/or any other cells that undergo MCP-1-dependent chemotaxis; stimulation of angiogenesis, or MCP-1-dependent stimulation of tumor growth and/or metastasis, measured as described herein or by any other assay known in the art; and/or any other physiological function carried out by an MCP-1 polypeptide molecule.

MCP-1 activity in a biological sample may be measured using one of the numerous techniques known in the art and/or described hereinbelow. For example, in addition to measuring MCP-1 activity by detecting or measuring: the relative amount of MCP-1 activity using one of the numerous receptor binding assays, chemotaxis assays, angiogenesis assays, tumor metastasis assays, or tumor survival assays described herein and/or known in the art, the relative level of MCP-1 activity may also be assessed by measuring the level of MCP-1 mRNA (e.g., by reverse transcription-polymerase chain reaction (RT-PCR) amplification, Northern hybridization, or dot-blotting); the level of MCP-1 protein (e.g., by ELISA or Western hybridization); the activity of a reporter gene under the transcriptional regulation of a MCP-1 transcriptional regulatory region (by reporter gene assay, e.g., employing beta-galactosidase, chloramphenicol acetyltransferase (CAT), green fluorescent protein (GFP), or luciferase as a reporter gene, as is well-known in the art); or the specific interaction of MCP-1 with another molecule (e.g., by the two-hybrid assay), e.g., a polypeptide known to interact with MCP-1 (e.g., a CCR2 receptor). For example, a compound that decreases the level of MCP-1 mRNA, protein, or reporter gene activity within a cell, a cell extract, or other experimental sample is a compound that inhibits the activity of MCP-1.

By “inhibiting MCP-1 activity” is meant decreasing MCP-1 activity (as defined above) by at least 10%, preferably by at least 20% or 30%, more preferably by at least 40%, 50%, or 60%, and most preferably by at least 70%, 80%, or 90%.

By “specifically interacts”, “specifically binds”, “specifically reacts with”, and similar terms is meant that an MCP-1 inhibitor (e.g., an anti-MCP-1 antibody) physically associates with its target molecule (e.g., an MCP-1 polypeptide) to inhibit MCP-1 activity. Preferably, the MCP-1 inhibitor does not substantially physically associate with other types of molecules.

By “expose” is meant to allow contact between an animal, cell, lysate or extract derived from a cell, or molecule derived from a cell, and a test compound.

By “treat” is meant to administer a compound or molecule that inhibits MCP-1 activity (i.e., but not limited to, a neutralizing antibody that specifically binds MCP-1) to a subject.

By “inhibit” is meant to slow, minimize, or prevent tumor growth or to slow, minimize, or prevent tumor metastasis in a subject by decreasing the activity of MCP-1 in the subject. Preferably, the inhibition of tumor growth or metastasis is by at least 20%, more preferably, by at least 30%, 40%, or 50%, still more preferably, by at least 60%, 70%, or 80%, even more preferably, by at least 90%, and most preferably, by 100%.

By “growth of a tumor” is meant that one or more cells within a tumor proliferate, resulting in more tumor cells and/or increased tumor mass.

By “metastasis of a tumor” is meant that one or more cells in a tumor breaks away from the tumor, and travels (e.g., via the lymphatic or circulatory system) to another location within the body. Once at the secondary location, the tumor cell may (but does not necessarily) undergo cell division to form a new tumor mass. For example, a breast carcinoma cell can break away from a primary tumor mass in the breast and metastasize to a lymph node, lung, or other location within the body. In turn, a cell within a secondary (or later generation) tumor mass may metastasize to yet a new site, and so on. The term “metastasis of a tumor” encompasses any type of tumor metastasis that is known in the art, and includes both micrometastasis and macrometastasis.

By “inhibiting metastasis” is meant to slow, minimize, or prevent tumor metastasis in a subject by decreasing the activity of MCP-1 in the subject, as described herein. Preferably, the inhibition of tumor metastasis is by at least 20%, more preferably, by at least 30%, 40%, or 50%, still more preferably, by at least 60%, 70%, or 80%, even more preferably, by at least 90%, and most preferably, by 100%, relative to the metastasis that would be observed in the absence of MCP-1-inhibitory therapy.

By “increasing the survival of a subject with a tumor” is meant prolonging the lifespan of the subject by decreasing the activity of MCP-1 in the subject, as described herein. Measuring from the time that MCP-1-inhibitory treatment is initiated, the survival of the subject is preferably increased by at least 1.25-fold, more preferably, by at least 1.5-fold, still more preferably, by at least 1.75-fold, and most preferably, by at least 2-fold, relative to the survival that would be observed in the absence of MCP-1-inhibitory therapy.

By “inhibiting growth of a tumor” is meant to slow, minimize, or prevent tumor growth (i.e., cell proliferation or an increase in tumor mass) in a subject by decreasing the activity of MCP-1 in the subject, as described herein. Preferably, the inhibition of tumor metastasis is by at least 20%, more preferably, by at least 30%, 40%, or 50%, still more preferably, by at least 60%, 70%, or 80%, even more preferably, by at least 90%, and most preferably, by 100%, relative to the tumor growth that would be observed in the absence of MCP-1-inhibitory therapy.

By “test compound” is meant a molecule, be it naturally occurring or artificially derived, that is surveyed for its ability to inhibit MCP-1 activity, by employing one of the assay methods described herein. Test compounds may include, for example, peptides, polypeptides, synthesized organic molecules, naturally occurring organic molecules, nucleic acid molecules, and components thereof.

By “sample” is meant an animal, a cell, a cell lysate, a cell extract, or a solution containing one or more molecules derived from a cell or cellular material (e.g. a polypeptide or nucleic acid), which is assayed as described above.

Therapeutic Strategies for Inhibition of MCP-1 Activity

Described herein are approaches for inhibiting tumor growth and/or metastasis and for increasing survival in a subject with cancer, by inhibiting MCP-1 activity. Therapeutic inhibition of MCP-1 activity may be used for treating and/or preventing recurrence or metastasis of any cancer whose growth or metastasis is directly or indirectly stimulated by the presence of MCP-1. Such cancers may include those in which MCP-1-secreting cancer cells are present, and/or those involving cancer cells that are stimulated to grow and/or metastasize by MCP-1. Moreover, in general, the methods of the invention may be used to treat and/or prevent recurrence of any cancer that grows and/or metastasizes via MCP-1-stimulated angiogenesis.

MCP-1 activity can be inhibited by administration of an MCP-1 inhibitor, e.g., any molecule that decreases, minimizes, or prevents interaction of MCP-1 with an MCP-1 receptor. Examples of MCP-1 inhibitors can include, but are not limited to, ligands or antibodies that specifically bind to MCP-1 or its receptor; functional nucleic acids (e.g., antisense nucleic acids, aptamers, ribozymes, and external guide sequences) that inhibit MCP-1 activity; and/or small molecule inhibitors of MCP-1 activity, as described herein, or as would be understood by one of ordinary skill in the art.

An MCP-1 inhibitor may inhibit MCP-1 activity by any biological mechanism. For example, an MCP-1 inhibitor may bind either to MCP-1 or to an MCP-1 receptor, thereby inhibiting the MCP-1/MCP-1 receptor interaction. Another mechanism by which an MCP-1 inhibitor may inhibit MCP-1 activity is by decreasing the amount of MCP-1 (or MCP-1 receptor) protein and/or nucleic acid, by, for example, increasing degradation of MCP-1 protein, MCP-1 mRNA, MCP-1 receptor protein, or MCP-1 receptor mRNA. This increase in degradation may be specific (e.g., via an MCP-1 inhibitor that specifically binds and targets an MCP-1 (or MCP-1 receptor) polypeptide or nucleic acid for destruction), or non-specific (e.g., by generally increasing protein or mRNA turnover). A molecule whose degradation is hastened by an MCP-1 inhibitor may be unassociated with a cell (e.g., an MCP-1 polypeptide molecule in the blood, lymph, or extracellular matrix), or the molecule whose degradation is hastened by an MCP-1 inhibitor may be cell-associated (e.g., a nucleic acid or polypeptide within a cell, or an MCP-1 receptor polypeptide at the surface of a cell). An MCP-1 inhibitor may also inhibit MCP-1 activity by decreasing transcription of an MCP-1 (or MCP-1 receptor) gene and/or translation of MCP-1 (or MCP-1 receptor) mRNA into an MCP-1 (or MCP-1 receptor) polypeptide. In sum, any molecule that inhibits MCP-1 activity, thereby inhibiting cancer growth and/or metastasis, and/or increasing survival of a subject with cancer, may be used in the methods of the invention.

Efficacy

Following administration of an MCP-1 inhibitor for treating cancer, as described below (e.g., administration of an antibody that inhibits MCP-1 activity), the efficacy of the MCP-1-inhibitory treatment can be assessed by evaluating the particular aspects of the medical history, signs, symptoms, and objective laboratory tests that are known to the skilled practitioner to be useful in evaluating the status of a patient with cancer or at risk for cancer, and therefore would be useful in evaluating the efficacy of therapeutic inhibition of MCP-1 activity in a patient with cancer. For instance, the size, number, and/or distribution of tumors in a subject receiving treatment may be monitored using standard tumor imaging techniques. A therapeutically-administered MCP-1 inhibitor that arrests tumor growth, results in tumor shrinkage, and/or prevents the development and/or metastasis of new tumors in a patient with cancer, in remission from cancer, or at risk for cancer, compared to the disease course that would occur in the absence of MCP-1 inhibitor administration, is an efficacious MCP-1 inhibitor for the treatment of cancer.

An efficacious MCP-1 inhibitor preferably increases the survival of a subject whose survival is threatened by a tumor whose growth and/or metastasis is stimulated by MCP-1 activity. Subject survivability is related to a number of things, including tumor type, tumor growth rate, and the propensity of the tumor to metastasize. The inhibition of MCP-1 activity interrupts and slows down tumor metastasis and the growth of the metastatic tumors once formed, as well as slowing down the tumor growth of the primary tumor.

While it is preferred that the subjects of this invention have identifiable tumors, it is also contemplated that the disclosed methods can be used to treat subjects who do not yet have identifiable tumors, but who would benefit from inhibition of MCP-1 activity, such as individuals who are in remission from cancer, have a family history of cancer, or who have one or more genetic predictors known to be associated with a higher than normal risk of developing cancer. One example of a subject who would benefit from MCP-1-inhibitory therapy is a female who has one or more strong predictors of developing breast cancer, such as family history and/or one or more genetic predictor known to place the woman at higher than normal risk for developing breast cancer. An efficacious MCP-1 inhibitor is one that decreases the likelihood that the subject will develop cancer, compared to the likelihood of developing cancer in the absence of MCP-1-inhibitory therapy.

Assays for Identifying Inhibitors of MCP-1 Activity

As described hereinbelow, MCP-1 activity, and inhibition thereof, is readily detectable using in vitro and in vivo assays. Therefore, such assays are useful for identifying compounds that are inhibitors of MCP-1 activity. For example, libraries of test compounds generated by combinatorial chemistry or other approaches may be screened for MCP-1-inhibitory activity by one or more of the many methods referred to herein and/or known in the art. In addition, any method that is, in the future, found to be a useful assay for MCP-1 activity, and/or inhibition thereof, may be used to identify MCP-1 inhibitors for use in the methods of the invention.

After candidate MCP-1 inhibitors are identified using one or more in vitro assays, MCP-1 inhibitory activity may be further confirmed using a secondary, in vivo assay for inhibition of MCP-1 activity. One example of such an assay involves administration of the candidate MCP-1 inhibitor to tumor-bearing mice (for example, but not limited to, the tumor-bearing mice described herein), followed by measurement of decreased tumor growth and/or metastasis, and/or increased survival. The skilled artisan will understand that the safety and efficacy of any candidate MCP-1 inhibitor is further tested using any animal model that would be considered, by one of ordinary skill in the art, to be appropriately used for such testing (e.g., a rodent, a rabbit, a dog, a pig, a primate), prior to testing in human clinical trials.

MCP-1 Inhibitors

The present invention provides inhibitors of MCP-1 activity for the treatment or prevention of cancer. These inhibitors interact with MCP-1 in a specific way to decrease, inhibit, or prevent one or more MCP-1 functions. For example, the inhibitors of this invention can interfere with the ability of MCP-1 to promote angiogenesis, resulting in a decrease in tumor growth, metastasis, and/or metastatic tumor growth. The inhibitors can either interact with MCP-1 polypeptide to bring about an inhibition of MCP-1 activity or the inhibitors can interact with MCP-1 nucleic acids to inhibit MCP-1 activity, through, for example, a decrease in MCP-1 production. The inhibitors can also interact with receptors of MCP-1 or MCP-1 receptor nucleic acids, reducing the interaction between MCP-1 and its cognate receptor or reducing the downstream effect of the MCP-1:receptor interaction.

The inhibitors can be any type of molecule capable of interacting with MCP-1, MCP-1 nucleic acid, an MCP-1 receptor, or an MCP-1 receptor nucleic acid in a way that inhibits its MCP-1 activity. For example, an MCP-1 inhibitor can be an antibody or a ligand (e.g., but not limited to, a peptide or small molecule) that binds MCP-1 or an MCP-1 receptor.

An MCP-1 inhibitor can also be a functional nucleic acid, such as an antisense molecule, an aptamer, a ribozyme or catalytic nucleic acid, a triplex forming molecule, or an external guide sequence (EGS). The antisense, triplex forming molecules, and EGSs are designed to target MCP-1 or MCP-1 receptor nucleic acid. The aptamers and catalytic nucleic acid molecules are designed to target either MCP-1 polypeptide, MCP-1 nucleic acid, MCP-1 receptor polypeptide, or MCP-1 receptor nucleic acid.

An MCP-1 inhibitor can also be a molecule that has been isolated from a combinatorial library that has been used to screen for MCP-1 binding function and MCP-1 inhibition function. Combinatorial libraries that contain numerous types of small molecules and methods of using these libraries are discussed below.

Antibodies

The term “antibodies” is used herein in a broad sense and includes both polyclonal and monoclonal antibodies. In addition to intact immunoglobulin molecules, also included in the term “antibodies” are fragments or polymers of those immunoglobulin molecules and humanized versions of immunoglobulin molecules or fragments thereof, as long as they directly or indirectly inhibit MCP-1 activity, as described herein.

Whenever possible, the antibodies of the invention may be purchased from commercial sources. The antibodies of the invention may also be generated using well-known methods. One of skill in the art will know how to choose an antigenic peptide for the generation of a monoclonal or polyclonal antibody that specifically binds an MCP-1 or MCP-1 receptor polypeptide. Antigenic peptides for use in generating the antibodies of the invention are chosen from non-helical regions of the protein that are hydrophilic. The PredictProtein Server (http://www.embl-heidelberg.de/predictprotein/subunit_def.html) or an analogous program may be used to select antigenic peptides to generate the antibodies of the invention. One of skill in the art will know that the generation of two or more different sets of monoclonal or polyclonal antibodies maximizes the likelihood of obtaining an antibody with the specificity and affinity required for its intended use (e.g., MCP-1-inhibitory therapy).

The antibodies are tested for their desired activity by known methods, in accordance with the purpose for which the antibodies are to be used (e.g., for immunotherapy; for further guidance on the generation and testing of antibodies, see, e.g., Harlow and Lane, Antibodies: A Laboratory Manual, Cold Spring Harbor Laboratory Press, Cold Spring Harbor, N.Y., 1988). For example, the antibodies may be tested in ELISA assays and/or in any assay for MCP-1 activity and inhibition thereof, as described herein. After their initial in vitro characterization, antibodies intended for therapeutic use are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

Monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies).

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fe fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRS) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fe), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

Functional Nucleic Acids

Methods are disclosed wherein MCP-1 activity is inhibited by contact between a functional nucleic acid and the MCP-1 polypeptide, MCP-1 nucleic acid, MCP-1-receptor, and/or the MCP-1-receptor nucleic acid. Also disclosed are methods wherein the MCP-1 activity is inhibited by delivering a functional nucleic acid to an MCP-1-producing cell within the subject. The functional nucleic acid molecule can specifically interact with the MCP-1 (or MCP-1 receptor) polypeptide and/or with the MCP-1 (or MCP-1 receptor) gene or mRNA.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, catalytic nucleic acid molecules (e.g., ribozymes), triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of MCP-1 or the genomic DNA of MCP-1, or with one or more DNA binding proteins that transcriptionally activate the MCP-1 gene, or they can interact with the MCP-1 polypeptide. Often functional nucleic acids are designed to interact with other nucleic acids based on sequence complementarity between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAse H-mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimizing antisense efficiency by identifying the most accessible regions of the target molecule. Exemplary methods are in vitro selection experiments and DNA modification studies using DMS and DEPC. It is preferred that antisense molecules bind the target molecule with a dissociation constant (k_{d}) less than 10⁻⁶. Antisense molecules can also bind with a k_{d} less than 10⁻⁸, less than 10⁻¹⁰, or less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with a k_{d} value from the target molecule of less than 10⁻¹² M. Aptamers can bind the target molecule with a k_{d} less than 10⁻⁶, less than 10⁻⁸, less than 10⁻¹⁰, or less than 10⁻¹².

Aptamers can bind the target molecule with a very high degree of specificity. For example, aptamers have been isolated that have greater than a 10,000-fold difference in binding affinities between the target molecule and another molecule that differ at only a single position on the molecule (U.S. Pat. No. 5,543,293). It is preferred that the aptamer have a kd with the target molecule at least 10-fold lower than the kd with a background binding molecule. An aptamer can have a k_{d} with the target molecule at least 100-fold, at least 1,000-fold, or at least 10,000-fold lower than the k_{d} with a background binding molecule. It is preferred when doing the comparison for a polypeptide for example, that the background molecule be a different polypeptide. For example, when determining the specificity of MCP-1 aptamers, the background protein could be thrombin. Representative examples of how to make and use aptamers to bind a variety of different target molecules can be found in the following non-limiting list of U.S. Pat. Nos: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acids. It is preferred that the ribozymes catalyze intermolecular reactions. There are a number of different types of ribozymes that catalyze nuclease or nucleic acid polymerase type reactions, which are based on ribozymes found in natural systems, such as hammerhead ribozymes (for example, but not limited to the following United States patents and publications: U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat), hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymenaribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific-cleavage of nucleic acids because recognition of the target substrate is based on the sequence of the target substrate. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5 5,877,022, 5,972,699, 5,972,704, 5,989,906, and 6,017,756.

Triplex-forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules can bind target regions with high affinity and specificity. Triplex-forming molecules can bind the target molecule with a k_{d} less than 10⁻⁶, less than 10⁻⁸, less than 10⁻¹⁰, or less than 10⁻¹². Representative examples of how to make and use triplex-forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

When the functional nucleic acid is targeting a nucleic acid rather than a polypeptide, depending on the type of functional nucleic acid used, it is relatively easy to isolated preferred sites for targeting. This is because most types of functional nucleic acids interact with the target nucleic acid through canonical and non-canonical base interactions and in addition have known specific sequence requirements that must go along with the base interactions. This is true for ribozymes, such as hammerhead and hairpin and for EGS molecules, as well as antisense and triplex-forming molecules. Computers can easily be programmed to perform algorithms that will search a given target sequence for the necessary sequence requirements for a specific type of functional nucleic acid. For example, the following algorithm to isolate hammerhead ribozymes for any target sequence can be found in the following PCT publications: WO 9858058 by Ludwig and Sproat, WO 9858057 by Ludwig and Sproat, and WO 9718312 by Ludwig and Sproat. For example, a typical hammerhead ribozyme prefers to cleaves an RNA molecule having the sequence N^{16.2}U^{16.1}H¹⁷ where N is any nucleotide, U is uridine, and H is an adenosine, cytidine, or uridine. (The numbering system simply refers to specific nucleotides in the standard configuration of a hammerhead ribozyme, Hertel et al., Nucl. Acids. Res., 20:3252 (1992)). Thus, a given sequence, such as MCP-1, or a sequence database, can be automatically searched to find target sites using the following representative algorithm:

i) find all NUH sequences where N is any nucleotide, U is uridine, and H is an adenosine, cytidine, or uridine;

ii) identify the potential base pairs surrounding this sequence;

iii) calculate the potential stem stabilities surrounding these sites; and

iv) sort sites according to stem stability.

There are many different configurations of hammerhead ribozymes that have different target sequence requirements, but each target sequence can be isolated in the same general way by changing the specific sequence searching requirements.

It is understood that the functional nucleic acids of the present invention can be made in a variety of ways, including enzymatically and synthetically. The functional nucleic acids can include a variety of nucleotides, nucleotide analogs, and nucleotide substitutes. Those of skill in the art will understand which types of modifications can be incorporated enzymatically and which must be incorporated synthetically.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10,1111-1118; Kabanov et al., FEBS Lett., 1990, 259, 327-330; Svinarchuk et al., Biochimie, (1993), 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates.

Methods for Isolating MCP-1 Inhibitors

Also disclosed are methods wherein the MCP-1 activity is inhibited by contacting an MCP-1 polypeptide molecule, MCP-1 nucleic acid, MCP-1-receptor molecule, and/or the MCP-1-receptor nucleic acid with an MCP-1 inhibitory molecule isolated by combinatorial chemistry or in vitro selection.

Combinatorial chemistry, includes but is not limited to, all methods for isolating small organic molecules or macromolecules that are capable of binding either another small molecule or another macromolecule. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100-nucleotide RNA pool, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dye. DNA molecules with such ligand-binding behavior have been isolated as well (Ellington and Szostak, Nature, 1992; Bock et al, Nature, 1992). Analogous strategies for isolating small organic molecules, proteins, antibodies, and other macromolecules having a desired characteristic (e.g., binding to a specific ligand) are known to those of ordinary skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which can bind MCP-1 that either have de novo activity or a modified activity. For example, phage display libraries have been used for a number of years to isolate peptides that interact with target molecules.

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can then be done to isolate functional peptides.

Once the selection procedure for peptide function is complete, traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selections. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that are attached to an acidic activation domain.

For example, a peptide or polypeptide fragment corresponding to an extracellular portion of a protein is attached to a DNA binding domain of a transcriptional activation protein, such as Gal 4. By performing the two-hybrid technique on this type of system, molecules that bind MCP-1 are identified. For example, full length MCP-1 as well as shorter fragments of MCP-1 can be attached to the DNA binding domain of a transcription activator. Libraries of peptides attached to an acidic activation domain can be screened for interaction with MCP-1 and subsequently analyzed for their inhibition effect in a variety of assays. Numerous commercial kits that include reagents necessary to perform the above screening method, such as the library of polypeptide fragments attached to the acidic activator and the cloning reagents for the DNA binding domain are available and the techniques and libraries can be produced de novo following the methods disclosed in U.S. Pat. Nos. 5,512,473, 5,580,721 5,580,736, 5,695,941, 5,780,262, 5,852,169, 5,938,321, 6,004,746, and 6,017,692.

Techniques for making and screening combinatorial libraries to isolate molecules that bind a desired target are well known to those of ordinary skill in the art. Using such methodology, those macromolecules that bind to or interact with the desired target are isolated and characterized. The relative binding affinity of these compounds are compared and optimum compounds are identified using competitive binding studies, which are well known to the skilled artisan.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. No. 5,698,685 and 5,506,337), sulfamides (U.S. Pat. No. 5,618,825), and benzodiazepines (U.S. Pat. No. 5,288,514).

Isolated molecules that bind MCP-1 can either be competitive inhibitors or non-competitive inhibitors with the natural binding partners of MCP-1, such as an MCP-1 receptor; conversely, isolated molecules that bind an MCP-1 receptor can either be competitive inhibitors or non-competitive inhibitors with the natural binding partners of the MCP-1 receptor, such as MCP-1.

Methods for Delivering Nucleic Acids that Inhibit MCP-1 Activity

Administration of nucleic acids that inhibit MCP-1 activity (e.g., functional nucleic acids and nucleic acids that encode MCP-1-inhibitory peptides and polypeptides), can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the MCP-1-inhibitory therapies described herein. Furthermore, these methods can be used to target certain diseases and cell populations by using the targeting characteristics of the carrier.

Transfer vectors can be any nucleotide construction used to deliver genes into cells, e.g., a plasmid or viral vector, such as a retroviral vector which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

Administration of MCP-1 inhibitors

The compositions and methods can be used therapeutically in combination with a pharmaceutically acceptable carrier. By “pharmaceutically acceptable carrier” is meant a material that is not biologically or otherwise undesirable, i.e., the material may be administered to an individual along with an MCP-1 inhibitor without causing any undesirable biological effects or interacting in a deleterious manner with any of the components of the pharmaceutical composition in which it is contained.

Pharmaceutical carriers are well-known in the art. These most typically are standard carriers for administration of vaccines or pharmaceuticals to humans, including solutions such as sterile water, saline, and buffered solutions at physiological pH.

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The MCP-1 inhibitors of the present invention can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Formulations for parenteral administration may include sterile aqueous solutions which may also contain buffers, diluents and other suitable additives.

The MCP-1 inhibitors of the invention are administered in an effective amount, using standard approaches. By “effective amount” is meant the amount of MCP-1 inhibitor that is useful for performing the stated function of the MCP-1 inhibitor, e.g., inhibiting tumor growth and/or metastasis and/or prolonging survival of a cancer patient. Effective dosages and schedules for administering MCP-1 inhibitors may be determined empirically, and making such determinations is routine to one of ordinary skill in the art. The skilled artisan will understand that the dosage of MCP-1 inhibitors will vary, depending upon, for example, the species of the subject the route of administration, the particular MCP-1 inhibitor to be used, other drugs being administered, and the age, condition, sex and extent of the disease in the subject. The dosage can be adjusted by the individual physician in the event of any counterindications. A dose of an MCP-1 inhibitor generally will range between about 100 μg/kg of body weight and 100 mg/kg of body weight. Examples of such dosage ranges are, e.g., about 100 μg-1 mg/kg, 1 mg/kg-10 mg/kg, or 10 mg-100 mg/kg, once a week, bi-weekly, daily, or two to four times daily.

For example, guidance for selecting an appropriate dose of an antibody (e.g., an MCP-1-inhibitory antibody) is found in the literature describing therapeutic use of antibodies. See, for example, Handbook of Monoclonal Antibodies, Ferrone et al. (eds.) Noges Publications, Park Ridge, N.J. (1985) Ch 22 and pp 303-357; Smith et al., Antibodies and Diagnosis in Human Therapy, Haber et al. (eds.) Raven Press, New York (1977) pp365-389.

### EXAMPLES

The following examples are intended to be purely exemplary of the invention and are not intended to limit the scope of the invention, since numerous modifications and variations thereto will be readily apparent to those of ordinary skill in the art.

### Example 1

Material and Methods

Chemokines and Antibodies

Recombinant human monocyte chemotactic protein-1 (MCP-1), vascular endothelial growth factor (VEGF), and basic fibroblast growth factor (bFGF), were purchased from Pepro Tech, Inc. (Rocky Hill, N.J.). Endothelial cell growth supplement (ECGS) was purchased from Sigma (St Louis, Mo.). Monoclonal anti-human CCR2 was kindly provided by Dr. Carlos Martinez (Centro National de Biotecnologia, Madrid, Spain), polyclonal antibody to human MCP-1 (Ab 279) was purchased from R&D Systems, Inc. (Minneapolis, Minn.). Rabbit IgG (Calbiochem, San Diego, Calif.) and mouse IgG (Coulter, Miami, Fla.) were used as the negative controls.

Cell Culture

Human umbilical cord vein endothelial cells (HUVECs) were isolated from neonatal umbilical cords. Human dermal microvascular endothelial cells (HMECS) were either obtained from Clonetics (Walkersville, Md.) or isolated from preputial skin (Bender et al., J. Clin. Invest. 79:1679-1688, 1987). Endothelial cell preparations were tested for their expression of CD31 and von Willebrand factor by flow cytometry, and preparations containing less than 2% contaminating cell types were selected for further studies. All endothelial cell types were cultured on collagen type I-coated plastic wells (Biocoat, Beckton Dickinson, Bedford, Mass.), in EGM medium (Clonetics, Walkersville, Md.) containing 5% FCS, VEGF (10 ng/ml), bFGF (10 ng/ml), glutamine (2 mM) and gentamycin (100 U/ml). All experiments were performed using subcultures between the second to seventh passage. The MDA-231 human breast carcinoma cell line was obtained from ATCC and grown in RPMI 1640 medium containing 5% FCS, glutamine (2 mM) and penicillin-streptomycin (100 U/ml).

Flow Cytometric Analysis

Indirect immunofluorescence was performed on HMECs and HUVECs by exposing cells to saturating amounts of mouse antibodies to human CCR2. Fluorescein-conjugated F(ab)2 fragments of goat anti-mouse (Sigma) diluted 1:50 was used as the secondary antibody. After staining, cells were analyzed using a FACScan flow cytometer (Becton Dickinson, Mountain View, Calif.).

Endothelial Cell Migration Assay

HMEC and HUVEC chemotaxis was performed using micro-Boyden chambers as described (Salcedo et al., Am. J. Pathol. 154:1125-1135, 1999). Briefly, polycarbonate filters of 5 μm pore size (Nucleopore, NeuroProbe, Cabin John, Md.) were coated with fibronectin (10 μg/ml) (Sigma) overnight at 4° C. Binding buffer containing 1.0 % BSA in RPMI 1640 with or without various amounts of MCP-1 was placed in the lower compartment of the chamber, and 0.5×10⁶ cells/ml resuspended in binding medium were then added to the upper compartment. The chambers were incubated for 4 h at 37° C. After the filters were removed, the upper surface was scraped, fixed with methanol, and stained with Leukostat (Fisher Scientific, Pittsburgh, Pa.). Membranes were analyzed using the BIOQUANT program (R & M Biometrics, Inc., Nashville, Tenn.), and the results were expressed as the mean number of migrated cells/ten fields at 10× magnification. For inhibitory assays, MCP-1 antibody was added together with MCP-1 in the lower compartment of the chamber. Each sample was tested by triplicates. Chemotaxis and inhibition of chemotaxis experiments were performed five times.

Rat Aortic Ring Assay

Rat aortic rings were prepared as previously described (Salcedo et al., Am. J. Pathol. 154:1125-1135, 1999). The thoracic and abdominal aorta was obtained from 100- to 150-g male Sprague-Dawley rats (Taconic, Germantown, N.Y.). Excess perivascular tissue was removed, transverse sections (1 to 2 mm) were made, and the resulting aortic rings were then washed in medium 199 (Gibco BRL, Life Technology, Grand Island, N.Y.). The rings were then embedded in Matrigel™ (Beckton Dickinson, Bedford, Mass.) in eight-well chamber slides (Nalge Nunc International, Milwaukee, Wis.) so that the lumen was parallel to the base of the slide. After the Matrigel™ gelled, serum-free medium (endothelial basal medium supplemented with antibiotics) with or without different concentrations of MCP-1 (1 to 100 ng/ml) was added to each well, and the slides were incubated at 37° C., with 5% CO₂, for 3 days (n=6 per dose). ECGS was used as the positive control at concentrations of 200 mg/ml. After the incubation period, the rings were fixed, stained and photographed. The ring assay was repeated two times.

Chick Chorioallantoic Membrane (CAM) Assay

Four ml of ovalbumin were removed from 3-day-old embryonated eggs (Truslow Farms, Charlestown, Md.). Thereafter, windows were opened for each egg, coated with tape, and eggs were incubated at 37° C. On day 10, five μl of distilled water containing different amounts of MCP-1 were applied in the center of quartered 13 mm diameter plastic coverslips (Thermanox, Nalge NUNC International) and allowed to dry for 10 min. Each coverslip was placed on the choriallantoic membrane of the chick and the eggs were incubated at 37° C. for 3 days. The assay was scored and photographed on the 13th embryonic day. EGF and water were used as positive and negative controls, respectively. Twenty eggs were used in total for each data point. A positive score for angiogenesis was made when vessels appeared to radiate from the spot in the coverslip to which the stimulant was applied. The scores are reported as a percentage of positive CAMs at each dilution.

In Vivo Matrigel™ Plug Angiogenesis Assay

Matrigel™ (9 mg/ml; 0.3 ml/mouse) alone or mixed with different concentrations of MCP-1 was injected subcutaneously into the flank of C57BL/6 mice. For angiogenesis inhibition, the mice were injected intraperitoneally with antibody to MCP-1 or control rabbit IgG (35 μg/mouse) on days 1, 3 and 6. On day 7, mice were sacrified and plugs were removed, fixed in 3.7% formaldehyde/phosphate-buffered saline (PBS), paraffin embedded, and Giemsa-stained slides were photographed. The experiment was repeated two times with eight mice per group in each experiment.

In Vivo Tumor Studies

CB-17 severe combined immune deficient (SCID) mice were used at 6 to 8 weeks of age and purchased from the animal production area (NCI-FCRDC, Frederick Md.). Animal housing and management were in accordance with the procedures outlined in the Guide for the Care and Use of Laboratory Animals (National Academy of Sciences, Institute of Laboratory Animal Resources, National Research Council, 1996), and the protocol used was approved by the NCI-FCRDC Animal Care and Use Committee. For survival experiments, SCID mice were injected intravenously (i.v.) with 20 μl of anti-ASGM1 (Wako Chemicals, VA) on day 0, and 3× 10⁻⁵ MDA231 human breast carcinoma cells were injected i.v. on day 1. Antibody to MCP-1 (Ab 279) (25 μg/mouse, 1 mg/kg) and control rabbit IgG were given intraperitoneally (i.p.) to the mice on days 4, 8, 12, 16, 20, 24 and 28. Ten mice were included in each group. Survival was monitored daily and moribund mice were euthanized. For experimental metastasis experiments, mice from both groups were sacrificed on the 35th day after i.v. injection of the tumor cells. Lungs were extracted and fixed in formalin. At this point few, if any, macrometastases were detected and micrometastases were quantitated. Histological sections were stained with hematoxylin and eosin, and tumor micrometastasis was quantitated using the Bioquant Program, counting the total tissue area per field 40× field (D1). The micrometastasis present within the same field were gated and the area within the gates was measured (D2). The metastatic index was calculated by the ratio D2/D1. A minimum of 20 fields was analyzed per slide and 8 mice were used per group in each experiment. The experiment was repeated two times. The survival experiment was repeated three times with ten mice per group in each experiment. Statistical analysis between the two groups was performed using Student's t-test (p<0.005).

Cell Proliferation Assay

MDA-231 were resuspended at 1×10⁶ cells/ml of proliferation medium (RPMI, 1% FCS, 2 mM glutamine and 100 U/ml penicillin 100 μg/ml streptomycin). 100 μl of the cell suspension/well were placed in 96 well plates and stimulated with different concentrations of MCP-1 in the presence or absence of antibody to MCP-1 (10 μg/ml). Plates were incubated at 37° C. in 5% CO₂ for 24, 48, or 72 hrs. To determine cell proliferation, cells were loaded with ³H-thymidine (0.5 to 1 μCi/well) 10 hrs before uptake determination. After the incubation plates were kept at −70° C. overnight and finally, the plates were thawed at room temperature, harvested, and ³H-thymidine incorporation was counted using a beta counter.

### Example 2

MCP-1 is Chemotactic for Human Microvascular and Umbilical Vein Endothelial Cells

Given earlier observations regarding the ability of certain ELR⁺ CXC chemokines and SDF-1α to induce neovessel formation, we first evaluated the capacity of human endothelial cells from either umbilical vein (HUVECs) or microvasculature (HMECs) to respond to MCP-1 by in vitro chemotaxis assay. We observed a dose-dependent chemotactic response for both cell types toward MCP-1. Table 1A shows the migration of HMECs and HUVECS toward different concentrations of MCP-1 (quantitated as the number of cells per 10× field); the maximal chemotactic response for each cell type was observed at 1 ng/ml of MCP-1.

To assess the specificity of the chemotactic response of endothelial cells toward MCP-1, we used a blocking polyclonal antibody to human MCP-1. Table 1B shows the results of a representative experiment (the assay was repeated five times) showing inhibition of the chemotactic responses of HMECs and HUVECS toward 1 ng/ml MCP-1 (the dose that induced maximal chemotaxis) by the mAB 279. This antibody specifically inhibited the chemotactic response of HUVECs and HMECs to MCP-1 when used at 10 μg/ml (Table 1B); (for Tables 1A and 1B, migration toward medium alone (basal migration) was subtracted from each data point; (*) p<0.001, (**) p<0.025). These data demonstrate that endothelial cells migrate in response to very low doses of MCP-1.

TABLE 1A
MCP-1 induces endothelial cell chemotaxis.

| | |
|---|---|
| | Average |
| MCP-1 concentration | number of migrated cells/field |

| (ng/ml) | HMECS | HUVECS |
|---|---|---|
| 0.1 | 22.6 ± 17.5 | 15.6 ± 18.8 |
| 0.5 | 95.6 ± 9.1 | 55.3 ± 15.1 |
| 1 | 128.6 ± 10.89 | 77.3 ± 11.5 |
| 5 | 103.6 ± 8.2 | 37.6 ± 8.4 |
| 10 | 85 ± 16.6 | 30.3 ± 15.1 |
| 50 | 36.3 ± 15.1 | 15.6 ± 9.1 |
| 100 | 18.3 ± 13.1 | 17.3 ± 15.1 |
| 500 | 27 ± 12.6 | 12.3 ± 4.8 |
| 1000 | 11.3 ± 10.4 | 11.6 ± 14.8 |

TABLE 1B
MCP-1-induced chemotaxis of endothelial cells is
inhibited by an anti-MCP-1 antibody.

Average
number of migrated cells/field

| Antibody | HMECS | HUVECS |
|---|---|---|
| none | 129 ± 13.4 | 74.3 ± 21.76 |
| IgG | 121 ± 21 | 84.9 ± 16.32 |
| Ab 279 (1 μg/ml) | 89.3 ± 19.16 | 48.2 ± 8.2 |
| Ab 279 (10 μg/ml) | 14.9 ± 6.3 | 10.4 ± 11.24 |

### Example 3

Human Microvascular and Umbilical Vein Endothelial Cells Express CCR2

The chemotactic response of HUVECs and HMECs to MCP-1 prompted us to investigate the expression of CCR2 (the receptor for MCP-1) on the surface of these endothelial cells by flow cytometric analysis. Cells were incubated with either IgG control or with an anti-CCR2 antibody. CCR2 was present on the cell surface of both HMECs and HUVECs. The mean fluorescence intensity was 62 (±14) for HMECs vs 37 (±9) for HUVECs, indicating that CCR2 is more abundantly expressed on the cell surface of HMECs than on HUVECs. The expression of CCR2 on HMECs was three-fold than that found on human monocytes.

### Example 4

MCP-1 Induces Angiogenesis In Vivo

To evaluate whether MCP-1 could exhibit angiogenic activities in vivo, we tested different concentrations of MCP-1 ranging from 1 to 1000 ng/ml using the chorioallantoic membrane (CAM) assay. MCP-1 at concentrations of 10 ng/ml and 100 ng/ml induced the typical radial formation of vessels characteristic of other well-known angiogenic factors such as EGF. The scores of the angiogenic response of MCP-1 were 80% and 50% positive CAMs when used at 10 ng/ml and 100 ng/ml, respectively. The negative control showed less than 15% positivity. No significant angiogenic responses were observed above the negative control level when MCP-1 was used at 1 ng/ml or at 1000 ng/ml. An inflammatory response, as indicated by an area with increased opacity on the coverslip, however, was also observed in association with the angiogenesis induced by MCP-1. These data demonstrate that MCP-1 has angiogenic effects in vivo.

### Example 5

Antibody to MCP-1 Inhibits the Angiogenic Effect of MCP-1 in the In Vivo Matrigel™ Plug Assay

We also evaluated the effect of MCP-1 using the in vivo matrigel plug assay. Mice were injected with Matrigel™ alone or with MCP-1 containing Matrigel™ subcutaneously in the flank. Histologic sections of the Matrigel™ plugs indicated a significant angiogenic effect induced by MCP-1 when used at concentrations of 10 or 100 ng/ml in contrast to Matrigel™ alone. We next asked whether the angiogenic effect of MCP-1 could be inhibited specifically by an antibody to MCP-1. Anti-MCP-1 significantly inhibited the angiogenesis induced by 100 ng/ml of MCP-1 to a level similar to that observed in the control Matrigel™ plugs lacking MCP-1. Moreover, injections of the control antibody did not inhibit this angiogenic effect. However, as observed using the CAM assay, an inflammatory reaction was also observed in the Matrigel™ plugs that contained MCP-1, which consisted predominantly of monocytes with few neutrophils. This inflammatory response was also inhibited by the MCP-1 antibody, but not by rabbit IgG control antibody. These data demonstrate that MCP-1 is angiogenic; however, it was not clear whether this effect was direct or indirect, e.g., mediated by the inflammatory cells.

### Example 6

MCP-1 Induces Rat Aortic Endothelial Cell Sprouting

Since, in our in vivo angiogenesis assays (the CAM and the Matrigel™ plug assays), MCP-1 angiogenic effects were accompanied by monocytic infiltration, we sought to investigate the possibility that the observed angiogenesis was leukocyte-dependent. We therefore tested the effect of MCP-1 using the ex vivo rat aortic ring-sprouting assay, which allows the detection of angiogenesis in the absence of an inflammatory response. Transverse sections of rat aorta tissue embedded in collagen were cultured with MCP-1 as described in the Methods section above, and thereafter examined for the degree of sprouting vessels. Cell culture medium and endothelial cell growth supplement medium was used as negative and positive controls, respectively. As shown in Table 2, MCP-1 stimulated numerous capillary sprouts at concentrations between 5 ng/ml (nmol/L) to 50 ng/ml. Thus, MCP-1 can induce endothelial cell sprouting at nanomolar concentrations from rat aortic rings in the absence of inflammatory infiltrates, indicating a direct effect in promoting angiogenesis.

TABLE 2
MCP-1 induces rat endothelial cell sprouting
in a dose dependent manner.

| | | | MCP-1 | | | | |
|---|---|---|---|---|---|---|---|
| Experiment | Aortic | | concentration (ng/ml) | | | | ECGS |
| number | rings | 0 | 0.5 | 5 | 50 | 500 | 100 ng/ml |

| | | | | | | | |
|---|---|---|---|---|---|---|---|
| 1 | 1 | − | − | + | ++ | + | +++ |
| | 2 | − | ++ | ++ | +++ | + | + |
| | 3 | − | + | − | ++ | + | ++ |
| | 4 | − | + | − | + | − | + |
| | 5 | − | + | ++ | + | − | + |
| | 6 | − | − | + | − | − | − |
| 2 | 1 | + | ++ | + | ++ | ++ | ++++ |
| | 2 | − | + | ++ | +++ | + | +++ |
| | 3 | + | − | − | +++ | − | ++++ |
| | 4 | − | − | + | ++ | − | ++ |
| | 5 | − | + | ++ | + | − | ++++ |
| | 6 | − | − | − | − | − | ++ |

Induction of endothelial cell sprouting by different concentrations of MCP-1 was compared to that observed in the presence of medium alone (negative control) or ECGS (positive control). Each concentration was tested by sextuples and the aortic rings were scored as follows: −: no sprouting, +: low sprouting levels, ++: 25% to 50% sprouting, +++: 50% to 75% sprouting, ++++: more than 75% sprouting. The scores of two separate experiments are shown.

### Example 7

Inhibition of MCP-1 Activity Enhances the Survival of SCID Mice Bearing MDA-231 Human Breast Carcinoma Cells

In view of our discovery of the direct angiogenic effect of MCP-1, we sought to evaluate the contribution of MCP-1 toward tumor growth. We therefore selected a human breast carcinoma cell line that produces MCP-1, MDA-231, to study the effect of an MCP-1 antibody on tumor growth. The MDA-231 cell line produced approximately 6,500 picograms of MCP-1/ml (as determined by ELISA) when cells were grown at a concentration of 0.5×10⁶ cells/ml of RPMI for 24 hrs. MDA-231 cells were then injected intravenously into SCID mice, as described in the Methods section above. As shown in Table 3, administration of MCP-1 antibody significantly increased the survival of SCID mice bearing MDA-231 carcinoma tumors, relative to mice treated with control antibody, in a statistically significant manner (p<0.024, assessed by Log-Rank Test; one representative experiment of three experiments is shown). Neither administration of exogenous MCP-1 nor antibodies to MCP-1 had an effect on the growth of MDA-231 cells in vitro (Table 4). While not wishing to be bound by theory, our results, taken together, suggest that inhibitors of MCP-1 activity prolong survival by inhibiting tumor-associated angiogenesis.

TABLE 3
Anti MCP-1 antibody enhances survival of SCID mice
bearing MDA-231 breast carcinoma cells.

Percentage of Survival

| Day | Control Antibody | Anti-MCP-1 Antibody |
|---|---|---|
| 1 | 100 | 100 |
| 5 | 100 | 100 |
| 7 | 100 | 100 |
| 12 | 100 | 100 |
| 13 | 100 | 100 |
| 16 | 100 | 100 |
| 31 | 100 | 100 |
| 31 | 90 | 100 |
| 39 | 90 | 100 |
| 39 | 80 | 100 |
| 43 | 80 | 100 |
| 43 | 70 | 100 |
| 43 | 70 | 100 |
| 43 | 50 | 100 |
| 45 | 50 | 100 |
| 45 | 40 | 100 |
| 46 | 40 | 100 |
| 47 | 30 | 80 |
| 49 | 30 | 80 |
| 49 | 30 | 70 |
| 52 | 30 | 70 |
| 52 | 20 | 70 |
| 53 | 20 | 70 |
| 53 | 10 | 70 |
| 55 | 10 | 70 |
| 55 | 10 | 60 |
| 67 | 10 | 60 |
| 67 | 0 | 50 |
| 72 | | 50 |
| 72 | | 40 |
| 78 | | 40 |
| 78 | | 30 |
| 89 | | 30 |
| 89 | | 20 |
| 129 | | 20 |
| 129 | | 0 |

TABLE 4
MCP-1 has no effect on the in vitro growth of MDA-231 cells.

³H-thymidine incorporation (CPM)

| MCP-1 | | Anti MCP-1 |
|---|---|---|
| (ng/ml) | No Antibody | (10 μg/ml) |
| 0 | 45,260 (±4,011) | 48,269 (±2,300) |
| 1 | 41,670 (±3,999) | 44,500 (±1,890) |
| 10 | 44,380 (±3,457) | 43,012 (±3,889) |
| 100 | 48,000 (±7,001) | 43,991 (±5,012) |
| The proliferation of MDA-231 cells was tested in the presence of different concentrations of exogenous MCP-1 and compared to that observed in the presence of medium alone. The effect of an MCP1 antibody on MDA-231 cellular proliferation was also tested, as measured by ³H-thymidine incorporation after 48 hours of MCP-1 stimulation. Each MCP-1 concentration was tested in triplicate. The average results of three experiments are shown ± s.e.m. Statistical analysis was | | |
| #performed using ANOVA relative to the medium alone. | | |

Analysis of the metastatic lesions in the lungs revealed that the experimental micrometastases therein were significantly smaller and lower in number when treated with the MCP-1 antibody than in the control antibody group. SCID mice bearing MDA-231 carcinoma tumors were treated with either control antibody or anti-MCP-1 antibody as described in Example 1 above. Grading analysis of the degree of lung metastasis, performed by calculating the total area invaded by tumor in each mouse, indicated that the group of mice treated with control antibody exhibited about 2.5 times more metastases than the group of mice treated with anti MCP-1. The metastatic index of control antibody-treated mice was 0.146 (SEM±0.027), whereas the metastatic index of anti-MCP-1 antibody-treated mice was 0.057 (SEM±0.011). These data demonstrate that the size and number of metastatic lesions formed in the presence of antibody to MCP-1 is reduced and survival is increased, indicating that MCP-1 has a role in tumor progression.

### Example 8

Anti-MCP-1 Antibody Inhibits Monocyte Chemotaxis Induced by Medium Conditioned by Melanoma or Breast Carcinoma Cell Lines

Based on the observation described in Example 7 above, i.e., that inhibition of MCP-1 inhibits growth and metastasis of MDA231 breast carcinoma cells in mice, it was next determined whether other breast carcinoma cell lines, as well as melanoma cell lines, secrete levels of MCP-1 comparable to those secreted by MDA231 cells. To answer this question, conditioned medium was prepared from various breast carcinoma and melanoma cell lines and the level of MCP-1 in each conditioned medium sample was measured. Each sample was then tested for the ability to induce monocyte chemotaxis in an MCP-1-dependent manner.

To prepare conditioned medium from breast carcinoma and melanoma cell lines, the wells of six-well plates were seeded with 1×10⁶ cells in one ml of RPMI-1640 plus 01.% FCS per well. The cells were cultured for 24 hours and the supernatants (conditioned medium) were collected. The conditioned medium was tested for the presence of MCP-1 using an ELISA kit from R&D Systems, Inc. (Minneapolis, Minn.). The breast carcinoma and melanoma cell lines used to prepare conditioned medium and the amount of MCP-1 present in each sample of conditioned medium are shown in Table 5 below.

TABLE 5
MCP-1 expression by tumor cell lines.

| Cell line | Origin | pg/ml |
|---|---|---|
| MDA 231 (ATTC catalog # HTB-26) | human breast | >2000 |
| | carcinoma | |
| MDA 436 (ATTC catalog # HTB-130) | human breast | >2000 |
| | carcinoma | |
| MDA 415 (ATTC catalog # HTB-128) | human breast | >2000 |
| | carcinoma | |
| MDA 330 (ATTC catalog # HTB-127) | human breast | >2000 |
| | carcinoma | |
| BT20 (ATTC catalog # HTB-19) | human breast | <15.6 |
| | carcinoma | |
| SKBR3 (ATTC catalog # HTB-30) | human breast | <15.6 |
| | carcinoma | |
| MCF-7 (ATTC catalog # HTB-22) | human breast | >2000 |
| | carcinoma | |
| T47D (ATTC catalog # HTB-133) | human breast | <15.6 |
| | carcinoma | |
| M14 (J. Invest. Dermatol. 105:248, 1995) | human | <2000 |
| | melanoma | |
| M21 (J. Invest. Dermatol. 105:248, 1995) | human | <31.2 |
| | melanoma | |
| DM150 (Cancer Res. 59:2724, 1999) | human | >2000 |
| | melanoma | |
| M10 (Biochem. Biophys. Res. Commun. | human | 1560 |
| 202:549, 1994) | melanoma | |

Supernatants from various human breast carcinoma and melanoma cell lines were tested for MCP-1 by ELISA. The data indicate the amount of MCP-1 produced by 0.5 million cells grown in 1 ml of media (expressed as pg/ml of MCP-1). Background of ELISA < 15 pg/ml.

Conditioned media containing the highest levels of MCP-1 were tested for the ability to stimulate MCP-1-specific chemotaxis of human monocytes. Monocyte chemotaxis assays were performed essentially according to the protocol set forth in Example 1 for the endothelial cell chemotaxis assays (Salcedo et al., Am. J. Pathol. 154:1125-1135, 1999), except that monocytes were seeded at 1×10⁶ cells/ml, and chemotaxis was allowed to proceed in the Boyden chambers for one hour. Recombinant human MCP-1 (10 ng/ml) was used as a positive control for monocyte chemotaxis, and goat anti-MCP-1 (10 mg/ml final concentration) was tested for its ability to inhibit monocyte chemotaxis stimulated by 30 μl of conditioned medium from various breast adenocarcinoma or melanoma cell lines. Tables 6 and 7 show the level of chemotaxis stimulated by each sample of conditioned medium, and the level of inhibition of chemotaxis resulting from the addition of an anti-MCP-1 antibody. No inhibitory effect of goat IgG was observed, indicating that the inhibitory effect of the anti-MCP-1 antibody was specific.

TABLE 6
Anti-MCP-1 antibody inhibits monocyte chemotaxis induced by
conditioned medium (CM) from breast carcinoma cell lines.

| | Average | |
|---|---|---|
| | number of migrating monocytes/20X field: | |
| Assay Condition | Minus antibody | Plus anti-MCP-1 antibody |
| Negative Control | 78 ± 46.8 | not done |
| MCP-1 | 719.7 ± 69.9 | 74.5 ± 3.4 |
| MDA231 | 675.6 ± 212.8 | 88.9 ± 94.8 |
| MDA436 | 547.8 ± 126.2 | 232.8 ± 121.2 |
| MDA415 | 458.5 ± 77.7 | 24.6 ± 36.7 |
| MDA330 | 764.5 ± 229.9 | 97 ± 56.4 |
| MCF7 | 564.4 ± 79.3 | 126 ± 12.8 |

TABLE 7
Anti-MCP-1 antibody inhibits monocyte chemotaxis induced by
conditioned medium (CM) from melanoma cell lines.

| | Average | |
|---|---|---|
| | number of migrating monocytes/20X field: | |
| Assay Condition | Minus antibody | Plus anti-MCP-1 antibody |
| Negative control | 44.3 ± 17.3 | not done |
| MCP-1 | 665.7 ± 98.8 | 35.8 ± 37.9 |
| M10 CM | 385 ± 79.8 | 106.7 ± 99.0 |
| M14 CM | 802.8 ± 234.8 | 131.1 ± 161.3 |
| DM150 CM | 713.4 ± 46 | 165 ± 22.8 |

Incorporation by Reference

Throughout this application, various publications are referenced. The disclosures of all these publications in their entireties are hereby incorporated by reference into this application in order to more fully describe the state of the art to which this invention pertains.

Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of inhibiting metastasis of a tumor in a subject, comprising inhibiting MCP-1 activity in the subject, thereby inhibiting metastasis of the tumor in the subject.

2. A method of inhibiting growth of a tumor in a subject, comprising inhibiting MCP-1 activity in the subject, thereby inhibiting growth of the tumor in the subject.

3. A method of increasing the survival of a subject with a tumor, comprising inhibiting MCP-1 activity in the subject, thereby increasing survival of the subject.

4. The method of claim 1, 2, or 3, wherein the tumor produces MCP-1.

5. The method of claim 1, 2, or 3, wherein the tumor is a solid tumor.

6. The method of claim 1, 2, or 3, wherein the tumor metastasizes in the subject.

7. The method of claim 1, 2, or 3, wherein the MCP-1 activity is inhibited by administering to the subject an antibody that specifically binds MCP-1 and inhibits MCP-1 activity.

8. The method of claim 7, wherein the antibody is a polyclonal antibody.

9. The method of claim 7, wherein the antibody is a monoclonal antibody.

10. The method of claim 1, 2, or 3, wherein MCP-1 activity is inhibited in the subject by inhibiting the interaction between MCP-1 and an MCP-1 receptor in the subject.

11. The method of claim 10, wherein the MCP-1 receptor is contacted with an antibody that specifically binds to the MCP-1 receptor, thereby inhibiting MCP-1 activity in the subject.

12. The method of claim 10, wherein the receptor is present on the surface of an endothelial cell.

13. The method of claim 10, wherein the antibody is a polyclonal antibody.

14. The method of claim 10, wherein the antibody is a monoclonal antibody.

15. The method of claim 1, 2, or 3, wherein the subject is a mammal.

16. The method of claim 15, wherein the mammal is a human.

17. The method of claim 1, 2, or 3, wherein the MCP-1 activity is stimulation of angiogenesis.

18. The method of claim 1, 2, or 3, wherein the MCP-1 activity is stimulation of tumor growth or tumor metastasis.

19. A method of identifying a compound that inhibits tumor growth or tumor metastasis, comprising:
a) exposing a sample comprising an MCP-1 polypeptide or an MCP-1 nucleic acid to a test compound; and
b) detecting a decrease in MCP-1 activity in the sample, wherein a decrease in MCP-1 activity in the sample identifies a compound that inhibits tumor growth or tumor metastasis.

20. A method of identifying a compound that inhibits tumor growth or tumor metastasis, comprising:
a) exposing a sample comprising an MCP-1 receptor polypeptide or an MCP-1 receptor nucleic acid to a test compound; and
b) detecting a decrease in MCP-1 activity in the sample, wherein a decrease in MCP-1 activity in the sample identifies a compound that inhibits tumor growth or tumor metastasis.

21. A method of identifying a compound that increases survival of a subject with a tumor, comprising:
a) exposing a sample comprising an MCP-1 polypeptide or an MCP-1 nucleic acid to a test compound; and
b) detecting a decrease in MCP-1 activity in the sample, wherein a decrease in MCP-1 activity in the sample identifies a compound that increases survival of a subject with a tumor.

22. A method of identifying a compound that increases survival of a subject with a tumor, comprising:
a) exposing a sample comprising an MCP-1 receptor polypeptide or an MCP-1 receptor nucleic acid to a test compound; and
b) detecting a decrease in MCP-1 activity in the sample, wherein a decrease in MCP-1 activity in the sample identifies a compound that increases survival of a subject with a tumor.
